(19) United States
(12) Patent Application Publication
(10) Pub. No.: US 2017/0367548 A1
(43) Pub. Date: Dec. 28, 2017

(54) HYGIENIC HAND DRYER AND ANTINFECTION SPRAYER

(71) Applicant: Fridolin Voegeli, Thalwil (CH)

(72) Inventor: Fridolin Voegeli, Thalwil (CH)

(21) Appl. No.: 15/192,313

(22) Filed: Jun. 24, 2016

(51) Int. Cl. A47K 10/48; A61L 2/00

(52) U.S. Cl. CPC A47K 10/48; A61L 2/0094; A61L 2202/15

(57) ABSTRACT

The present invention has been derived from the ubiquitous automatic hand dryer, but including also a spraying system to sanitize the dried hands. The hollow spraying & drying chamber with hundreds ventilator outlets and multiple spray nozzles functions as a closed-loop, air-recycling system: The drying air is pressed towards the surface of the inserted hands in a down-stream angle; this downstream being enforced by the ventilator aspirating the air from the bottom outlet of the spraying & drying chamber. In contrast to the ubiquitous automatic hand dryer, the casing is closed, completely on the side walls and leaving only small openings at the higher-end side, large enough to insert the hands and suck-in air from above. With this closed chamber and the air-stream from outside above downward along the hand's surface, any emission of air-water droplets as aerosols, upward and to the environment, against the incoming air-stream, is impossible.

### FIELD OF THE INVENTION

The present invention relates to automatic hand dryer of the ubiquitous model patented, designed and sold by Mitsubishi first and then by its copyists, used in restrooms and cloakrooms, in private and public places: We insert the hand into the device, at a vertical or more natural downward angle, and when pulling the hand out, air-blades wipe the water down and drain it from the hands surface. In contrary the present invention asks for a careful drying-out of the water from the sprayed-on ANTINFECTION matrix.

### BACKGROUND OF THE INVENTION

Washing the hands and protecting them against new contamination, by applying a long-time protecting media is a GMP (Good Manufacturing Practice) standard in food production and catering, in pharmaceutical production and application, in semiconductor miniaturization and electronics assembly, etc. It is one of the basic measures who enabled today's high quality, high yield, zero defect, long-time guarantees, even from mass productions in developing countries.

Washing the hands and protecting them against next, imminent contamination shall be the new Best Practice in medicine, combating the proliferation of infectious diseases, especially of the prevailing hospital-acquired-infections (HAI), also called nosocomial infections: The WHO, in the foreword to her 2009 “Guidelines on Handhygiene in Health Care” is lamenting:
“Healthcare-associated infections affect hundreds of millions of patients worldwide every year. Infections lead to more serious illness, prolong hospital stays, induce long-term disabilities, add high costs to patients and their families, contribute to a massive, additional financial burden on the healthcare system and, critically, often result in tragic loss of life.” [From many studies we know, that the number of “tragic loss of life” from those infections brought home from the clinics is several 10 millions per year!]

Our patent document U.S. Pat. No. 8,961,873 “Antinfection Protecting Healthcare Workers to Prevent Spreading of Communicable and Nosocomial Infections” describes a new method of ANTINFECTION: Applying a long-time persistent antiseptic spray, fortified with polymers that build a protective matrix on the surface of the hands. Using a sophisticated, automatic device that incorporates also a (hot) air dryer, to dry the water from the polymer matrix.

The 30 years of “WHO Disinfection Guidelines” have proven, that washing and drying the hands and subsequently disinfecting them, is a catastrophe: It is killing everything on the (still clean) surface, also the self-defence mechanisms of the skin (pH, fat, resident bacteria). The next transient germs will find ideal conditions for undisturbed landing, invasion and reproduction.

As the next years shall prove, washing and drying the hands without subsequent sanitizing them (10 sec max) with a long-term protection is non-sense; the “complete” procedure shall be: Washing the hands, drying the water, plus applying long-time sanitizer and drying again.

Searching thru the Internet and patent libraries for a suitable hand dryer as prototype for our first ANTINFECTION spraying machine we came across patent U.S. Pat. No. 7,555,209 “Hand Dryer”, marketed today as Model V300 by the Spanish company Veltia: It is the only known hand dryer using multiple (some 300) ventilation outlets, trying to “control” the local drying, instead of just blowing off and away all water residues from the hands, with a air-jet stream of more than 100 m/sec.

The patent is a good base for any later design concepts; it also references some 10 other, implemented patents and defines their weaknesses. Most interesting, in our evaluation, was still the first modern patent of U.S. Pat. No. 5,459,944, filed 1993 by Mitsubishi: It contained a hollow form of the dry chamber that was just cosmetically changed by later copyists. The chamber was open only at the top and had two outlets for wiped-off water at the bottom; the side walls of the casing were closed. The later copyists did not get the reason behind this: So most actual designs with their highest speed air-blades work as “water+dirt+bacteria centrifuge”, blowing out at the open top (also U.S. Pat. No. 7,555,209) and on the more or less low side-wall ports (like old-timer cars side-wall before the time of closable doors). Most active marketer of such “open-sided” hand dryers, Dyson claims a reduction of consumables (paper towels), but does not tell us, that most operational tests show a 100 to 1000-times heavier load of mostly infectious water-droplets in the rest-rooms' air, then before.

These water-centrifuging is a No-go for any sanitizing, and any hands-cleaning-only! It shall never be tolerated by the Environmental and Hygiene authorities for any ANTINFECTION (and soon also no more for simple toilet hand-washing and drying).

### SUMMARY OF THE INVENTION

We therefore had to invent a hygienic hand dryer fitting into our ANTINFECTION Sprayer, pictured in our U.S. Pat. No. 8,961,873, or a hygienic hand dryer that had an ANTINFECTION Sprayer built-in. After some design studies, we decided to merge an adequate hand dryer's drying chamber with our ANTINFECTION Sprayer's spraying chamber: The resulting spraying & drying key sub-system can now be built-into our ANTINFECTION Sprayer (at a 30° downward angle) or into an ubiquitous hand dryer (e.g. at a 60° angle).

The Hygienic Hand Dryer of the present invention has some constructive particularities aimed at reducing the load of blown-out water-droplets in the environment, especially upwards, towards the face of the user, as depicted in the following illustrative figures.

### BRIEF DESCRIPTION OF THE DRAWINGS

FIG. 1 shows a cross-section of the new spraying & drying chamber of the new Hygienic Hand Dryer & ANTINFECTION Sprayer, ready to be mounted into a ubiquitous hand dryer.

FIG. 2 shows the outlines of the spraying chamber from our ANTINFECTION device that shall also contain the elements for the drying function.

FIG. 3 shows a finished implementation in an attractive design to go into the public space, e.g. into airport terminals. It also shows that the hollow chamber may have two distinct cavities, one for each hand. (For the special looks of the clamshell the designer didn't give up the side-wall openings. We had to close them with glass, barely visible in the photo.)

### DETAILED DESCRIPTION OF THE PREFERRED EMBODIMENTS

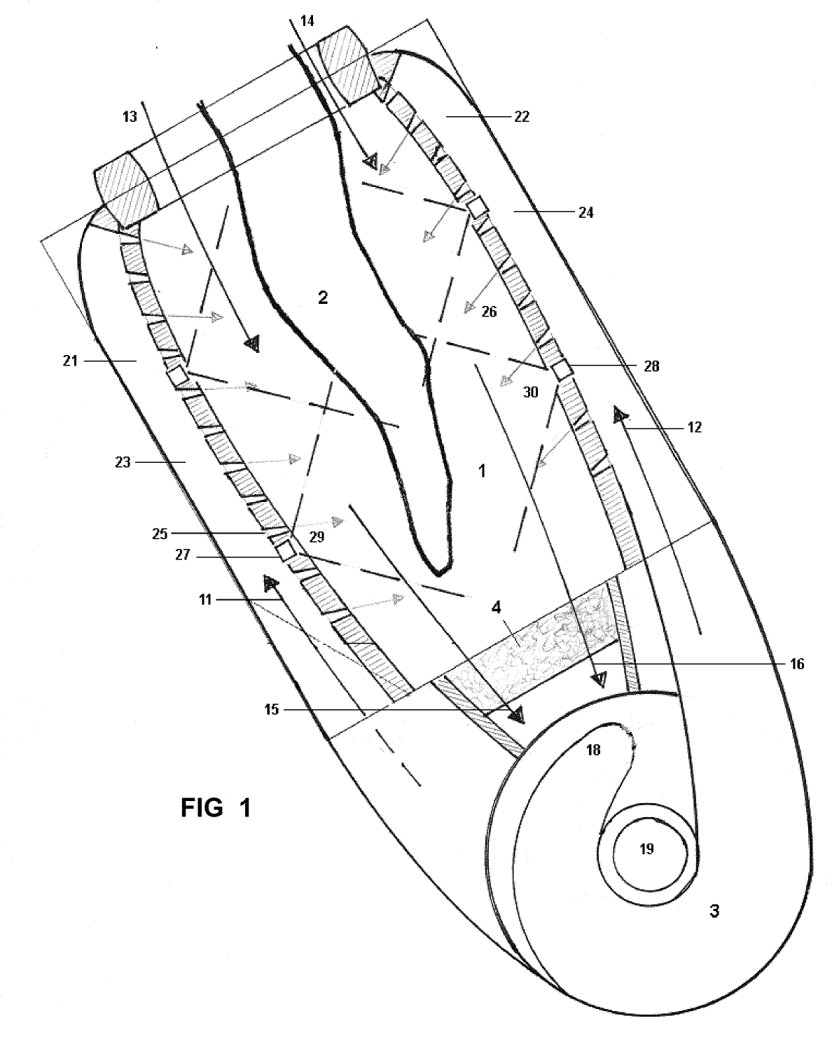

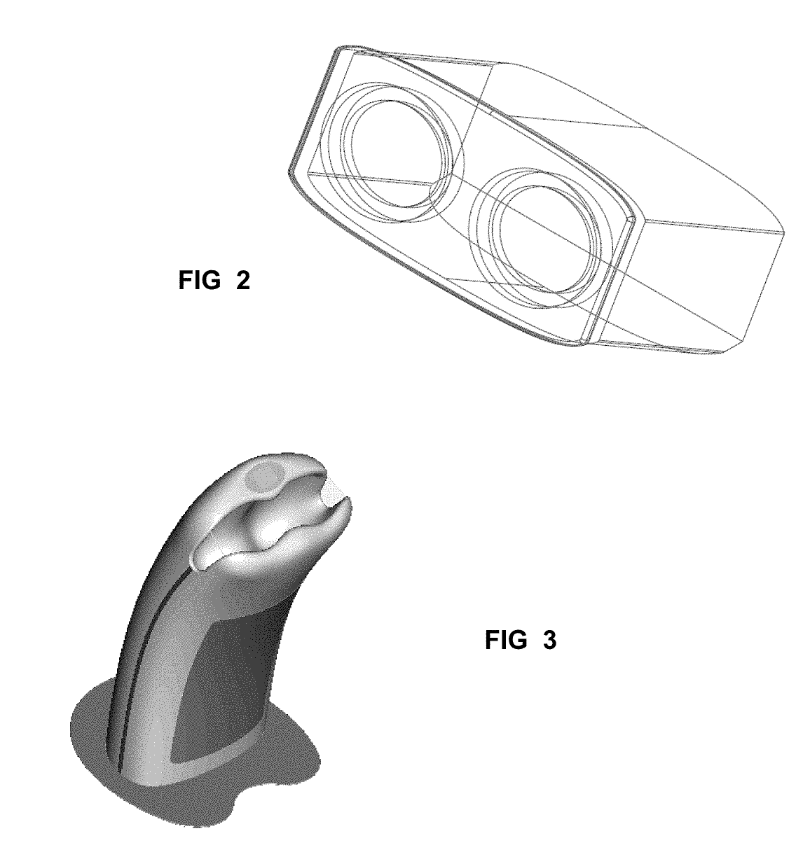

In FIG. 1 the new spraying & drying chamber (1) has been cut thru the middle of the right hand (2) inserted some 10 cm off the middle line of the device. (The chamber's inner dimensions and its key elements are to scale, some 1:2. The “attached” ventilator (3), filter-box (4) and connecting air-ducts are not, they have to be sized, grouped and finally fixed in the available casing.)

The top wall and the bottom each have a half of the ventilating elements (21, 22) accurately fitted: These inner walls have on their double concave surfaces hundreds of orifices, as first proposed in V300 by Veltia, all operating simultaneously. These orifices guide the air, pushed upwards (11, 12) by the ventilator, deflect it from the air-ducts (23, 24) and project it thru narrowing openings (25) onto the entire surface of the hands. The resulting direction angle of these air blasts (26) is slightly downwards, increased by the top-down stream of fresh air (13, 14) being aspirated by the ventilator (15, 16).

These ventilating elements are highly sophisticated constructions, optimized for best pressure and flow distribution and deflection, assembled in 3 to 4 layers. Materials and forms are also selected for minimal friction, to avoid turbulences that would significantly reduce air speed and laminar flow. For more details see U.S. Pat. No. 7,555,209.

In free spaces between the ventilation orifices some 10-20 miniature spray nozzles (27, 28) eject their very small volumes of ANTINFECTION Spray (29): Some 3-5 mL for 2 hands, i.e. some 200-400 μL per nozzle; sprayed in less than 0.5 sec and dried in less than 7 sec.

In FIG. 1 the arrows (11, 13, 15) on the bottom side and the arrows (12, 14, 16) on the top side show the resulting main air-streams and their directions: The only aspiration into the nearly closed system is from above, thru the remaining slit between the user's wrist and the (rubber-cushioned) minimal top opening of the chamber (some 10×12 cm oval). The air flows from the hand finally (15, 16) go thru the filter-box (17) into the 2 aspiration ports (18, 18b) of the 2 turbines (19, 19b), one for the bottom side and one for the top side, and back-upwards (12, 11). There is only one exit from the closed-system, for the drained water plus some air, thru the drain holes (20, 20b) at the bottom corners of the chamber.

This new closed-loop ventilation running in the closed-chamber operates in two different drying modes: When DRAINING ONLY, as in the old ubiquitous hand dryer, the key requirement is highest possible speed of the air jets, in order to break-off the cohesive water-film on the skin surfaces and to make the water droplets fall down by gravity. Now, when first SPRAYING the hands with the ANTINFECTION protection matrix, it is more important to “press the spray on the skin”, with an air stream of lower speed, but higher pressure and eventually higher temperature, to DRY OUT the water in the undisturbed polymer-matrix. (The higher temperature can in addition increase the efficacy of some antiseptic ingredients.)

During DRAINING ONLY, for drying washed wet hands before spraying, the drained water falls towards the bottom of the chamber (30) to be collected in a drain tank, to be disposed of by authorized personal doing refill and maintenance service. When SPRAYING (29, 30) there should be only minimal spray falling down into the bottom of the chamber (30), the main part of the water surplus in the matrix is transported away by the (hot) air-stream during the last phase of DRY OUT.

1. An ANTINFECTION Sprayer comprising, in addition to all control and service related components, as key-subsystem a hygienic spraying & drying chamber, for inserting the hands parallel, in a natural down-direction and treating them in this still position.

2. An automatic hand dryer of the ubiquitous type, comprising, in addition to all control and service related components, as key-subsystem a hygienic drying chamber, for inserting the hands parallel, in a natural down-direction and treating them in this still position.

3. The hygienic spraying & drying chamber of claims 1 and 2 implemented as hollow chamber, including in the top and bottom wall each, ventilating elements with hundreds of directed orifices blowing air on the entire surface of the hand.

4. The hygienic spraying & drying chamber of claims 1 and 2 implemented as hollow chamber with only minimal openings at the higher end, the hands-insert side, to aspire additional air from outside above into the ventilation system, in order to prevent any emission of water or spray droplets as aerosols against this air-stream, to the outside above.

5. The openings for hand insertion of claim 4 implemented with flexible borders to be controlled, more closed after hand insertion, during spraying & drying.

6. The two ventilating wall elements of claim 3 being connected to a turbine each, building a closed-loop ventilation system, with intermediate filters and with only two small outlets for water and a little air at the lowest corners of the chamber.

7. The ventilating wall elements of claim 3 integrating in addition to the air-jet orifices multiple miniature valves and nozzles to spray controlled the entire surface of the hand.
